(19) United States
(12) Patent Application Publication
(10) Pub. No.: US 2018/0362914 A1
(43) Pub. Date: Dec. 20, 2018

(54) MINIATURIZED ROTATING BIOREACTORS

(71) Applicant: Matthew D. Millard, Cambridge, MA (US)

(72) Inventor: Matthew D. Millard, Cambridge, MA (US)

(21) Appl. No.: 15/625,698

(22) Filed: Jun. 16, 2017

(51) Int. Cl. C12M 3/04; C12M 1/00; C12M 1/04; C12M 1/26; C12M 3/00

(52) U.S. Cl. CPC C12M 27/12; C12M 23/38; C12M 23/44; C12M 29/04; C12M 33/18; C12M 23/24

(57) ABSTRACT

A rotating bioreactor. A bioreactor base has a cover support section positioned within the base. Air holes extend through the base and connect to the cover support section. An air permeable membrane is positioned on top of the cover support section and covers the air holes. A base cover has a chamber tube extending through the base cover. The base cover is inserted onto the base and is supported by the cover support section. A sealing mechanism is positioned between the base cover and membrane. A septum is attached to the base cover. A cell containment chamber is formed within the rotating bioreactor and is bordered by the membrane, the sealing mechanism, the chamber tube and the septum. The cell containment chamber is air permeable but water and fluid impermeable.

The present invention relates to bioreactors, and in particular, to rotating bioreactors.

### BACKGROUND OF THE INVENTION

Adverse drug reactions account for 7% of hospital admissions and, furthermore, 15% of inpatients experience a drug-related adverse reaction during each hospitalization. It is estimated that 0.3% of hospitalized patients die from complications due to adverse reactions from administered pharmaceutical drugs accounting for an estimated 100,000 deaths per year in the US [Lauschke, V. M., et al., Novel 3D Culture Systems for Studies of Human Liver Function and Assessments of the Hepatotoxicity of Drugs and Drug Candidates, Chem Res Toxicol, 2016. 29(12).]—approximately the same number of Americans that die of Alzheimer's. Pharmaceutical drug-induced liver injury is the most common adverse drug reaction and liver toxicity is the most common reason for cessation of drug development or withdrawal of a drug from the market. [Kaplowitz, N., Idiosyncratic drug hepatotoxicity, Nat Rev Drug Discov, 2005. 4(6).] Candidate drugs undergo costly and thorough pre-clinical testing in both in vitro models and animal models, following the proper FDA guidelines. Currently kidney and liver testing is extremely expensive and difficult to accomplish correctly. The failure rate and cost is unacceptably high.

For example, the toxicity of fialuridine was missed by testing of hepatocytes in 2D cultures, as well as testing in rats, mice, dogs, and cynomolgus monkeys. The drug was administered to 15 patients of whom seven developed liver toxicity. Five died and two were saved by liver transplantation. [McKenzie, R., et al., Hepatic failure and lactic acidosis due to fialuridine (FIAU), an investigational nucleoside analogue for chronic hepatitis B, N Engl J Med, 1995. 333(17)]. However, recent studies have shown that fialuridine toxicity could have been detected with better in vitro models that are just now emerging. Four other drugs recently withdrawn from the market for liver toxicity, including Terfenadine (Seldane®), Mibefradil (Posicor®), Astemizole (Hismanal®), and Cisapride (Propulsid®), also passed current in vitro and clinical safety testing. [Information obtained from Food and Drug Administration at its website at: www.fda.gov/drugs/developmentapprovalprocess/developmentresources/druginteractionslabeling/ucm115979.htm.]

Prior art rotating wall vessels are known. For example, U.S. Pat. No. 6,730,498 discusses a method for production of functional proteins including hormones by renal cells in a three dimensional co-culture process responsive to shear stress using a rotating wall vessel. However, prior art rotating wall vessels have drawbacks. The currently available rotating wall vessels are prohibitively costly in terms of both equipment and requirement for hepatocytes (liver cells) and are not amenable to industrial scale toxicity testing.

### Electrical Rollers

Electrical rollers are known in the prior art. For example, FIG. 12 shows a perspective view of electrical roller 70. An electrical motor (not shown) operates to turn circular roller bars 72 in either a clockwise or counterclockwise rotation. Items place on top of roller bars 72 will then rotate in an opposite direction. For example, it is common to place hot dogs on top of roller bars 72. Heat is then applied underneath roller bars 72 and the hot dogs are then cooked in an even manner. For example, FIG. 12 shows hot dog electrical roller Central Model#: 40K-019 Brand: Value Series Mfg Part#: 62020, available from Central Restaurant Products in Indianapolis, Ind.

What is needed is a commercially viable, low cost, better rotating bioreactor.

### SUMMARY OF THE INVENTION

The present invention provides a rotating bioreactor. A bioreactor base has a cover support section positioned within the base. Air holes extend through the base and connect to the cover support section. An air permeable membrane is positioned on top of the cover support section and covers the air holes. A base cover has a chamber tube extending through the base cover. The base cover is inserted onto the base and is supported by the cover support section. A sealing mechanism is positioned between the base cover and membrane. A septum is attached to the base cover. A cell containment chamber is formed within the rotating bioreactor and is bordered by the membrane, the sealing mechanism, the chamber tube and the septum. The cell containment chamber is air permeable but water and fluid impermeable.

### BRIEF DESCRIPTION OF THE DRAWINGS

FIG. 1 shows a perspective view of a preferred embodiment of the present invention.

FIGS. 2A-6D show views of components of a preferred embodiment of the present invention.

FIG. 7 shows an exploded view of a preferred embodiment of the present invention.

FIGS. 8-11 show a preferred method for connecting the components of a preferred embodiment of the present invention.

FIG. 12 shows a prior art electrical roller.

FIGS. 13-14 show a preferred method for using a preferred embodiment of the present invention.

### DETAILED DESCRIPTION OF THE PREFERRED EMBODIMENTS

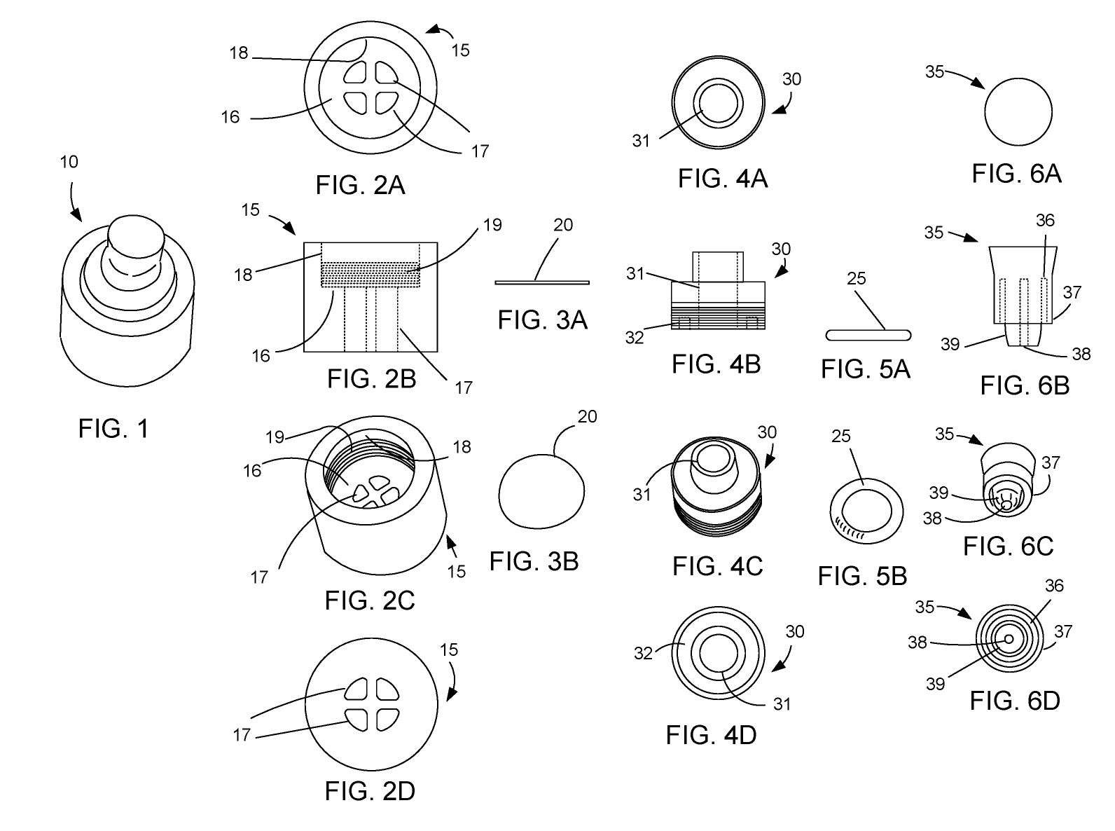

FIG. 1 shows a perspective view of miniaturized rotating bioreactor 10. In a preferred embodiment, bioreactor 10 is utilized to maintain differentiated hepatocytic organoids in suspension under conditions of physiologic shear stress.

Some features of bioreactor 10 are listed below. The utilization of bioreactor 10 exposes hepatocytes to controlled low levels of shear stress that mimic blood flow and are important for maintenance of key hepatocyte functions. Bioreactor 10 also maintains functional hepatocytes for extended periods of time, e.g. days to weeks, allowing for chronic toxicity testing. Bioreactor 10 has its design optimized rapidly and economically using 3-D printing and/or injection molding. Multiple prototypes can be produced within hours, tested in the laboratory, and modified in iterative cycles. In a preferred embodiment, bioreactor 10 is produced in an expedited fashion through the use of 3-D printed injection mold casts to allow rapid facile inexpensive production of test vessels. Bioreactor 10 is very economical in terms of the number of hepatocytes that may be cultured and the equipment required compared to currently available rotating wall vessels. Utilization of bioreactor 10 greatly simplifies media loading, cell inoculation, drug addition, and sample collection. Also, bioreactor 10 supports co-localization of other cell types of differing densities, such as Kuppfer cells, into the organoids where they can modulate the hepatocytes' functions.

### Components of the Bioreactor

### The Base

FIG. 2A shows a top view, FIG. 2B shows a side view, FIG. 2C shows a perspective view and FIG. 2D shows a bottom view of base 15. In one preferred embodiment, base 15 is approximately 9/16 inches in height and ¾ inches in diameter. These dimensions may be varied, as appropriate. Base 15 may be fabricated from glass, hard plastic, silicone or other suitable material. Cover support section 16 is horizontally positioned within base 15, as shown. Air holes 17 extend through the bottom of base 15 and connect to cover support section 16. Interior base wall 18 includes ridged interior surface 19. Ridged interior surface 19 has ridges that provide for an improved grip of base cover 30 when base cover 30 is inserted into base 15. Ridged interior surface 19 has teeth that mesh with similar teeth on base cover 30 positioned at appropriate angles to allow for a span-fit connection locking base cover 30 onto base 15. Other types of locking connection mechanisms are also preferred and can also allow for an optimum fit between base 15 and base cover 30. These other locking connection mechanisms include a threaded connection, a press fit connection, interlocking cogs, interlocking teeth and other similar connection mechanisms.

### Membrane

FIG. 3A shows a side view and FIG. 3B shows a perspective view of membrane 20. Membrane 20 is seated within base 15 by being positioned on top of chamber support section 16. Membrane 20 covers and seals holes 17. Membrane 20 is permeable to air but keeps water inside chamber 25. Membrane 20 may be fabricated from a variety of materials. In one preferred embodiment it is fabricated from plastic. In another preferred embodiment it is fabricated from rubber.

### O-Ring

FIG. 5A shows a side view and FIG. 5B shows a perspective view of O-ring 25. O-Ring 25 fits into groove 32 of base cover 30 and functions to seat base cover 30 onto membrane 20 and over holes 17. In a preferred embodiment O-ring 25 is fabricated from a fluoropolymer. It may also be fabricated from Teflon® or nylon.

### Base Cover

FIG. 4A shows a top view, FIG. 4B shows a side view, FIG. 4C shows a perspective view and FIG. 4D shows a bottom view of base cover 30. Base cover 30 is preferably fabricated from glass. Base cover 30 includes chamber tube 31 that extends through base cover 30. Ridged exterior surface 32 is at the bottom of base cover 30 and meshes with ridged interior surface 19 of base 15 for a secure fit. Groove 32 receives O-ring 25 to seat base cover 30 onto membrane 20 and over holes 17.

### Septum

FIG. 6A shows a top view, FIG. 6B shows a side view, FIG. 6C shows a perspective view and FIG. 6D shows a bottom view of septum 35. Septum 35 is preferably fabricated from flexible soft rubber. Septum 35 includes groove 36, flexible grasping rim 37, insertion section 39 and interior passage 38. In a preferred embodiment septum 35 is placed over chamber tube 31 to provide an upper seal for base cover 30. Septum 35 prevents water or other fluid from leaving base cover 30.

### Piecing Together the Bioreactor

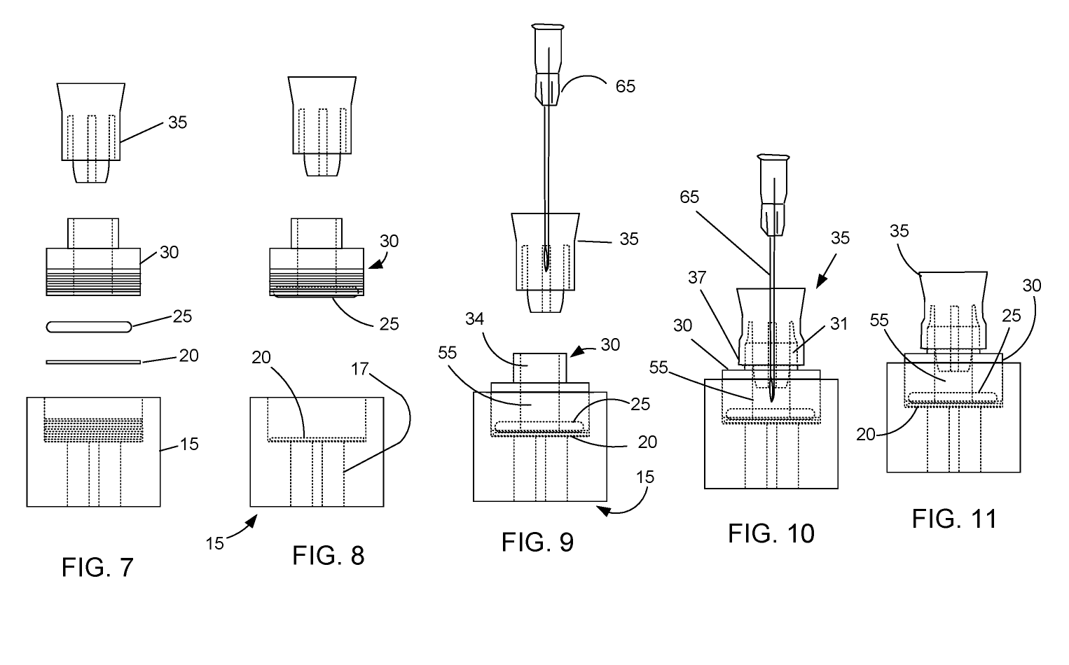

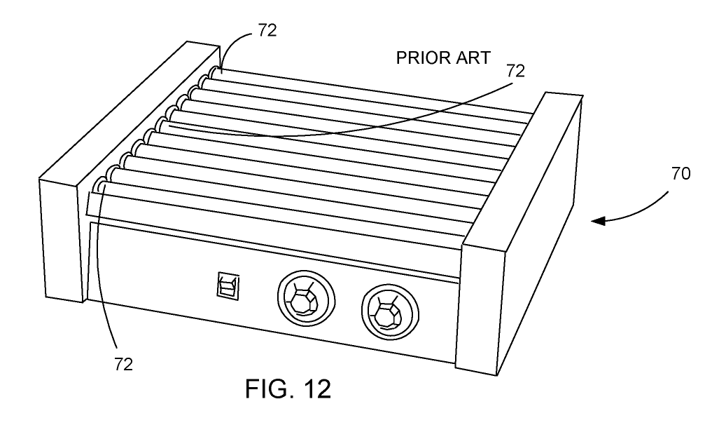

FIG. 7 shows an exploded view of septum 35, base cover 30, O-ring 25, membrane 20, and base 15.

In FIG. 8, membrane 20 has been placed inside base 15 to cover and seal holes 17. Membrane 20 is permeable to air but will prevent fluid and water from flowing through holes 17. O-ring 25 has been press fitted into groove 32 of base cover 30 (FIG. 4D).

In FIG. 9, base cover 30 has been lowered onto base 15 and base cover 30 has been locked into place by utilization of ridged exterior surface 32 and ridge interior surface 19 (see above discussion). O-ring 25 forms a seal between base cover 30 and membrane 20. Cell containment chamber 55 is then created, as shown. A pipette is then preferably used to transfer a cell solution through large bore opening 34 into cell containment chamber 55. A drug may be then added to the cell solution in cell containment chamber 55 to test its effect on cells in the cell solution. A small gage needle (for example, twenty-gage needle 65) is then poked through septum 35 to allow a passageway for air so that air may be bled out as the septum is applied.

In FIG. 10, septum 35 has been press fit on top of base cover 30. Grasping rim 37 encircles the top of chamber tube 31 to provide a seal on the top of base cover 30 that seals cell containment chamber 55. Air trapped in cell containment chamber 55 is quickly bled out through needle 65.

In FIG. 11, needle 65 has been pulled out of septum 35. Septum 35 is flexible rubber and closes after removal of needle 65 so that septum 35 is impermeable after the removal of needle 65. Septum 35 provides a seal to the top of cell containment chamber 55. In a preferred embodiment cell containment chamber 55 is able to receive and hold a cell solution for drug testing. As stated above, the cell containment chamber is air permeable through membrane 20. Fluid and water are prevented from flowing out by membrane 20, O-ring 25, base cover 30 and septum 35.

### Utilization of the Rotating Bioreactor

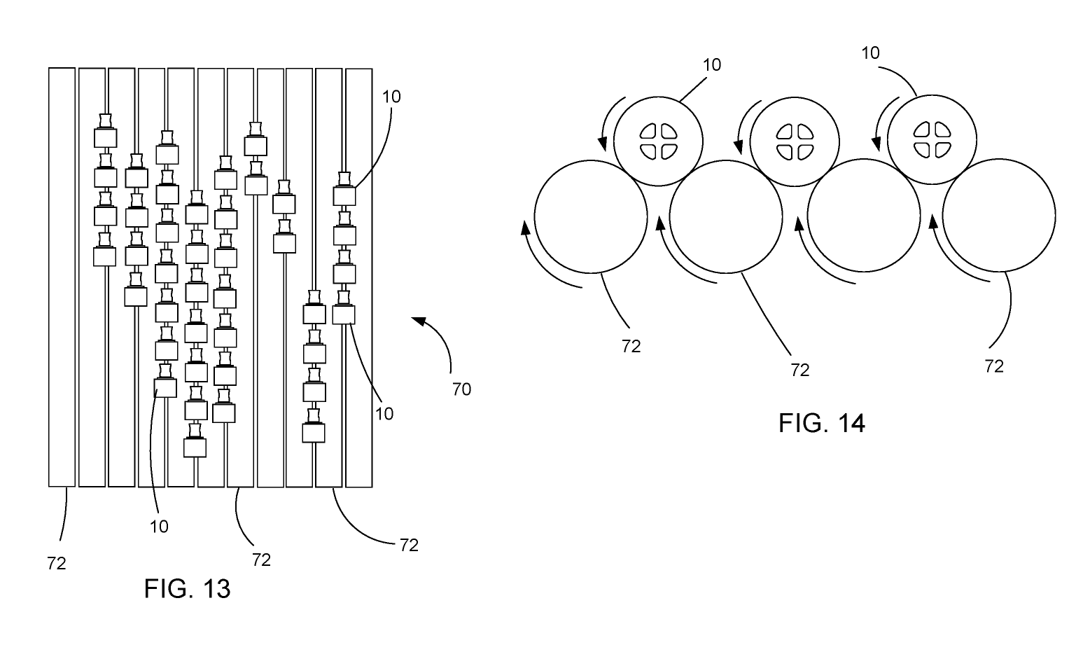

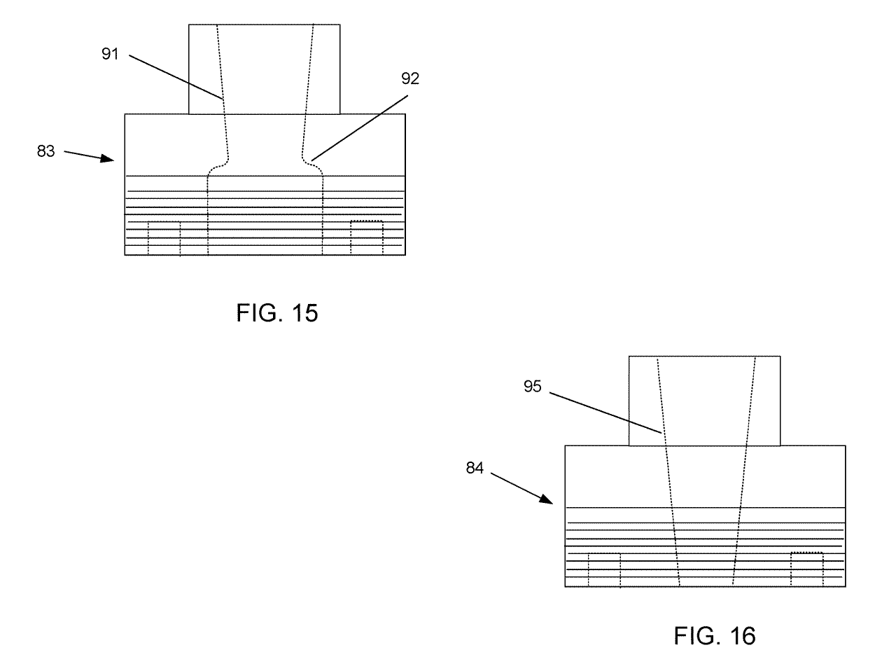

In a preferred embodiment, rotating bioreactors 10 are placed onto roller bars 72 of electric roller 70 (FIG. 13). Rotating bioreactors 10 each contain cell solution having cells for evaluation. The cells are contained in a cell solution in cell containment chamber 55 (FIG. 11). Bioreactors 10 are then rotated on roller bars 72 (FIGS. 13 and 14) for extended periods of time to allow for chronic toxicity testing of cells within cell containment chamber 55. While being rotated, rotating bioreactors 10 expose the cells in cell containment chamber 55 to controlled low levels of shear stress that mimic blood flow and are important for maintenance of key cell functions. In a preferred embodiment, electric roller 2 is a controlled environment electric roller and is enclosed in an environment that can be customized and maintained while bioreactors 10 are being rotated and while the cells are being tested. For example, it is important that humidity, temperature and roller speed are carefully monitored and maintained to prevent damage to the cell solution and bioreactor components.

### Improved Base Cover

FIGS. 15 and 16 show improved base cover 83 and 84, respectively. Base cover 83 includes sloped wall 91 and smooth bend 92 as features that prevent the trapping of air within cell containment chamber 55. Because of the features, air is trapped by neither capillary action nor shaped pockets. Likewise, base cover 84 includes sloped wall 95 as a feature that prevents the trapping of air within cell containment chamber 55. Because of sloped wall 95, air is trapped by neither capillary action nor shaped pockets.

Although the above-preferred embodiments have been described with specificity, persons skilled in this art will recognize that many changes to the specific embodiments disclosed above could be made without departing from the spirit of the invention. For example, a drug that is being tested may be added to a cell solution in cell containment chamber 55 at any time, even days after bioreactor 10 has been sealed with septum 35. A needle may be utilized to poke through septum 35 to add a drug to the cell solution in cell containment chamber 55 after bioreactor 10 has been sealed. Also, even though the above preferred embodiments discussed the utilization of O-ring 25 it should be understood that other sealing mechanisms may be utilized besides an O-ring. For example, a flat gasket may also be utilized. Also, it should be noted that bioreactor 10 may be either hand assembled and filled or machine assembled and filled through automation. Also, it was described above that a function of membrane 20 is to allow air to flow into cell containment chamber 55. In another preferred embodiment other materials bordering cell containment chamber 55 are permeable to air. For example, in one preferred embodiment base cover 30 is permeable to air. In such an embodiment, the utilization of membrane 20 can be avoided because it is no longer needed. Therefore, the attached claims and their legal equivalents should determine the scope of the invention.

What is claimed is:

1. A rotating bioreactor, comprising:
A. a bioreactor base comprising a cover support section positioned within said base and comprising air holes extending through said base and connecting to said cover support section,
B. a membrane positioned on top of said cover support section and covering said air holes,
C. a base cover comprising a chamber tube extending though said base cover, wherein said base cover is inserted into said base and is supported by said cover support section,
D. a sealing mechanism positioned between said base cover and said membrane,
E. a septum attached to said base cover,
wherein a cell containment chamber is formed within said rotating bioreactor and is bordered by said membrane, said sealing mechanism, said chamber tube and said septum, wherein said cell containment chamber is air permeable but water and fluid impermeable.

2. The rotating bioreactor as in claim 1, wherein said bioreactor base and said base cover are locked together by utilization of a locking connection mechanism.

3. The rotating bioreactor as in claim 1, further comprising a needle extending through said septum to allow a passageway for air.

4. The rotating bioreactor as in claim 1, wherein at least one said bioreactor is positioned on an electrical roller to rotate said at least one bioreactor.

5. The rotating bioreactor as in claim 4, wherein said at least one bioreactor is a plurality of bioreactors.

6. The rotating bioreactor as in claim 4, wherein aid at least one bioreactor is positioned on roller bars of said electrical roller to rotate said at least one bioreactor.

7. The rotating bioreactor as in claim 1, wherein said base cover further comprises an O-ring attachment groove and said sealing mechanism is an O-ring attached to said O-ring attachment groove.

8. The rotating bioreactor as in claim 1, wherein said bioreactor is hand assembled and filled.

9. The rotating bioreactor as in claim 1, wherein said bioreactor is machine assembled and filled through automation.

10. The rotating bioreactor as in claim 4, wherein said electric roller is positioned within an enclosed area where humidity, temperature and roller speed are monitored and maintained to prevent damage to cell solution and said bioreactor.

11. The rotating bioreactor as in claim 4, wherein said electric roller is a controlled environment electric roller and wherein said rotating bioreactor is housed on said controlled environment electric roller.

12. The rotating bioreactor as in claim 1, wherein said base cover comprises features to prevent trapping of air within said cell containment chamber.
